(19) United States
(12) Patent Application Publication
(10) Pub. No.: US 2026/0305920 A1
(43) Pub. Date: Oct. 8, 2026

(54) MEDICAL DEVICES AND RELATED SYSTEMS AND METHODS

(71) Applicant: Boston Scientific Medical Device Limited, Galway (IE)

(72) Inventors: Yao CHEN, Shanghai (CN); Linshanzi PAN, Shanghai (CN); Shibo WANG, Shanghai (CN); Mengxiang LUO, Shanghai (CN); Cheng ZHANG, Shanghai (CN)

(73) Assignee: Boston Scientific Medical Device Limited, Galway (IE)

(21) Appl. No.: 19/640,506

(22) Filed: Apr. 7, 2026

(30) Foreign Application Priority Data
Apr. 8, 2025 (CN) 202510435481.7

(51) Int. Cl. A61B 1/00; A61B 1/06; A61B 1/307

(52) U.S. Cl. CPC A61B 1/00154; A61B 1/00119; A61B 1/00131; A61B 1/0661; A61B 1/307

(57) ABSTRACT

Medical devices useful to perform medical procedures such as aspiration are described. The medical device may comprise a sheath defining a lumen extending to a distal end of the sheath. The lumen may be configured to slidably receive a medical instrument. A pressure sensor may be embedded within or coupled to an outer surface of the sheath. A plurality of light sources may be arranged about a perimeter of the distal end of the sheath, and the pressure sensor may be proximal to the plurality of light sources.

### CROSS-REFERENCE TO RELATED APPLICATIONS

This application claims the benefit of priority to Chinese Application No. 202510435481.7, filed on Apr. 8, 2025, which is incorporated by reference herein in its entirety.

### TECHNICAL FIELD

Aspects of the disclosure generally relate to devices, systems, and methods for medical sheaths and medical scopes. Some aspects relate to medical devices, systems, and methods for using a sheath including several components and a shaft of a medical device.

### BACKGROUND

Medical devices, such as scopes and sheaths, are commonly inserted into a patient’s body to perform a therapeutic and/or diagnostic procedure. During procedures like endoscopy or ureteroscopy, medical professionals rely on illumination sources integrated into the scope to visualize target tissue, deliver energy to tissue, or administer fluids. Some medical scopes also incorporate pressure sensors, suction mechanisms, or other tools, which occupy space within the shaft. The internal space within the shaft is limited in the ability to serve multiple functions, such as transporting medical instruments, providing irrigation or suction at the target site, and housing internal components such as electrical wiring. As medical devices integrate additional tools and functionality, the space allowed for working channels may become constrained, limiting space for additional instruments like forceps, needles, and brushes.

### SUMMARY

Examples of this disclosure relate to, among other things, medical devices, and systems useful for medical procedures, including medical devices and systems with the capability to provide aspiration during a medical procedure. For example, the medical device may comprise a sheath defining a lumen extending to a distal end of the sheath, the lumen configured to slidably receive a medical instrument, and a pressure sensor embedded within or coupled to an outer surface of the sheath, and a plurality of light sources arranged about a perimeter of the distal end of the sheath. The pressure sensor may be proximal to the plurality of light sources.

According to some aspects, each light source of the plurality of light source sources may comprise a light-emitting diode (LED) or an optical fiber. The distal end of the sheath may include a tapered portion, e.g., wherein the plurality of light sources may be disposed on the tapered portion. The plurality of light sources may include at least four light sources, e.g., at least ten light sources optionally arranged at regular intervals. In some aspects, the lumen may have a diameter ranging from about 10 mm to about 15 mm. The lumen may extend an entire length of the sheath. According to some aspects, each light source of the plurality of light sources may include an optical fiber extending through a wall of the sheath. Additionally, or alternatively, the pressure sensor may be configured to trigger an alarm when measuring a pressure outside the medical device that exceeds a predetermined threshold.

Further, for example, the medical device may comprise a handle coupled to or integrated with the sheath, and the handle optionally may include a suction port in fluid communication with the lumen. The suction port may include an aperture configured to control pressure in the medical device when the suction port is coupled to a source of vacuum. The sheath may be flexible and configured to navigate through a subject, such as through a urinary system of a subject. The pressure sensor may be adjacent to at least one light source of the plurality of light sources.

This disclosure also includes systems comprising the medical device described above and/or elsewhere herein. For example, the system may comprise the medical device and the medical instrument. For example, the medical instrument may be slidably disposed in the lumen. Further, for example, the medical instrument does not include a light source, e.g., a scope that does not include a light source. In some examples, the scope is a ureteroscope.

This disclosure also includes a medical device comprising a sheath defining a lumen extending to a distal end of the sheath, the lumen configured to slidably receive a ureteroscope. A pressure sensor may be embedded within or coupled to an outer surface of the sheath. The medical device may include a plurality of light sources, e.g., at least four light sources, arranged about a perimeter of the distal end of the sheath. The pressure sensor may be proximal to the plurality of light sources. The medical device may include a handle coupled to or integrated with the sheath, e.g., the handle including a suction port in fluid communication with the lumen.

According to some aspects, each light source of the plurality of light sources may comprise a light-emitting diode (LED) or an optical fiber. The distal end of the sheath may include a tapered portion, and the plurality of light sources may be disposed on the tapered portion. In some aspects, the lumen may have a diameter ranging from about 10 mm to about 15 mm.

This disclosure also includes a system comprising a medical device comprising a sheath defining a lumen extending an entire length of the sheath, the lumen configured to slidably receive a medical instrument, a pressure sensor embedded within or coupled to an outer surface of the sheath, and a plurality of light sources. The plurality of light sources may be arranged about a perimeter of a distal end of the sheath. The pressure sensor may be proximal to the plurality of light sources. The system may include a medical instrument, e.g., a scope that does not include a light source.

### BRIEF DESCRIPTION OF THE FIGURES

The accompanying drawings, which are incorporated in and constitute a part of this application, illustrate exemplary aspects that, together with the written description, explain the principles of this disclosure. The figures depict exemplary aspects according to this disclosure, as follows:

FIG. 1 depicts a side-view of an exemplary medical device comprising a sheath, according to some aspects of this disclosure;

FIG. 2 depicts an enlarged view of the distal end of the medical device of FIG. 1;

FIG. 3 depicts a cross-sectional view of the distal end of the medical device of FIG. 1;

FIG. 4 depicts a side view of an exemplary medical system comprising the medical device of FIG. 1 and a medical instrument;

FIG. 5A depicts an end view of the medical device and medical instrument of the system of FIG. 4; and

FIG. 5B depicts an end view of another exemplary medical system comprising the medical device of FIG. 1 and another medical instrument.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

Although aspiration sheaths and scopes such as ureteroscopes are referenced herein for illustration purposes of medical devices and medical instruments, it will be appreciated that the disclosure encompasses any suitable medical device or instrument configured to help an operator to access and view internal body anatomy of a subject and/or to deliver suction, irrigation, and/or medical instruments such as, for example, biopsy forceps, graspers, baskets, snares, probes, scissors, capture/retrieval devices, lasers, and other tools, into the subject’s body. The medical devices and instruments herein may be inserted into a variety of body lumens and/or cavities, such as, for example, the urinary tract or gastrointestinal tract. It will be appreciated that, unless otherwise specified, bronchoscopes, duodenoscopes, endoscopes, gastroscopes, endoscopic ultrasonography (“EUS”) scopes, colonoscopes, ureteroscopes, bronchoscopes, laparoscopes, cystoscopes, aspiration sheaths, catheters, or any other suitable delivery device may be used in connection with the features described herein.

Aspects of the disclosure describe exemplary medical systems, instruments, and devices, which may provide relatively more space to accommodate, for example, tools or to facilitate suction. The medical devices herein may include a handle and a sheath defining a lumen. Medical systems in accordance with this disclosure may include a medical device and a medical instrument insertable into the lumen of the sheath of the medical device. The sheath of the medical device may include sensor, such as a pressure sensor, and at least one light source (e.g., optical fiber or LED), such that the medical instrument need not include these components. The medical devices and systems herein may provide for additional space for a working channel of the medical instrument.

Reference will now be made in detail to examples to help illustrate aspects of the present disclosure through the accompanying drawings. Wherever possible, the same reference numbers will be used throughout the drawings to refer to the same or like parts.

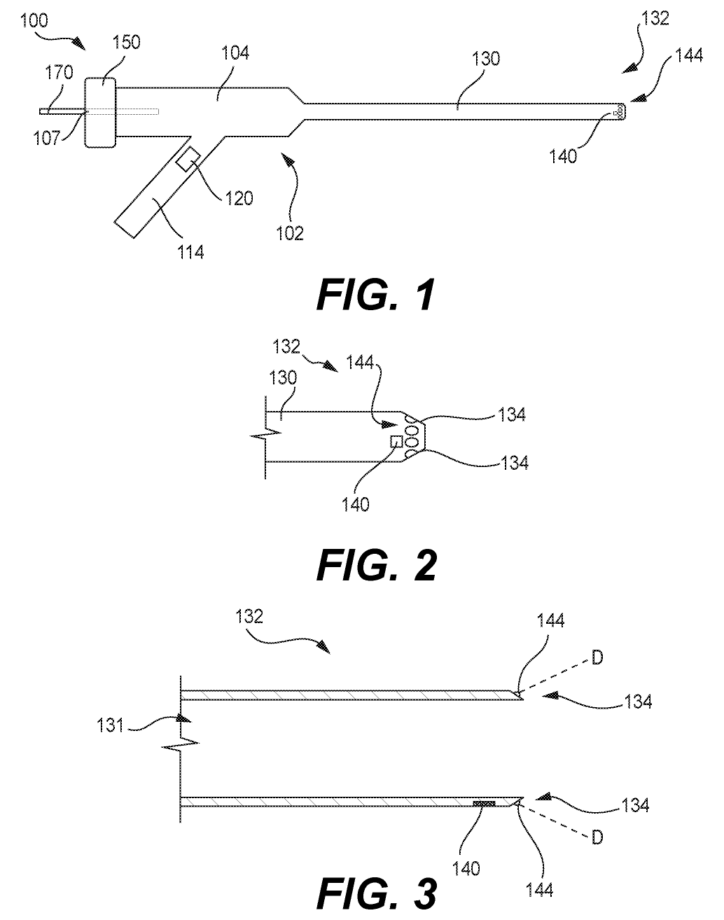

FIG. 1 depicts an exemplary medical device 100 according to aspects of this disclosure, medical device 100 may be configured to receive a shaft 170 of a medical instrument 160, such as a scope. Medical device 100 may be configured to help a user (e.g., a medical professional) to deliver medical instruments to access internal areas of a subject’s body, e.g., to perform medical diagnoses and/or treatments on the subject, with the ability to provide suction and/or irrigation. Medical device 100 includes a handle 102 that may be coupled to or integral with a sheath 130. Thus, for example, handle 102 may be in fluid communication with a lumen or working channel of sheath 130.

Medical device 100 may be an aspiration sheath or a similar device configured to deliver medical instruments to internal areas of a subject's body for diagnosis and/or treatment, while providing suction and/or irrigation capabilities. Sheath 130 may be configured to navigate through a body lumen, such as a urinary tract of a subject. Sheath 130 may define a lumen 131 that extends the length of sheath 130 to a distal end 132 of sheath 130.

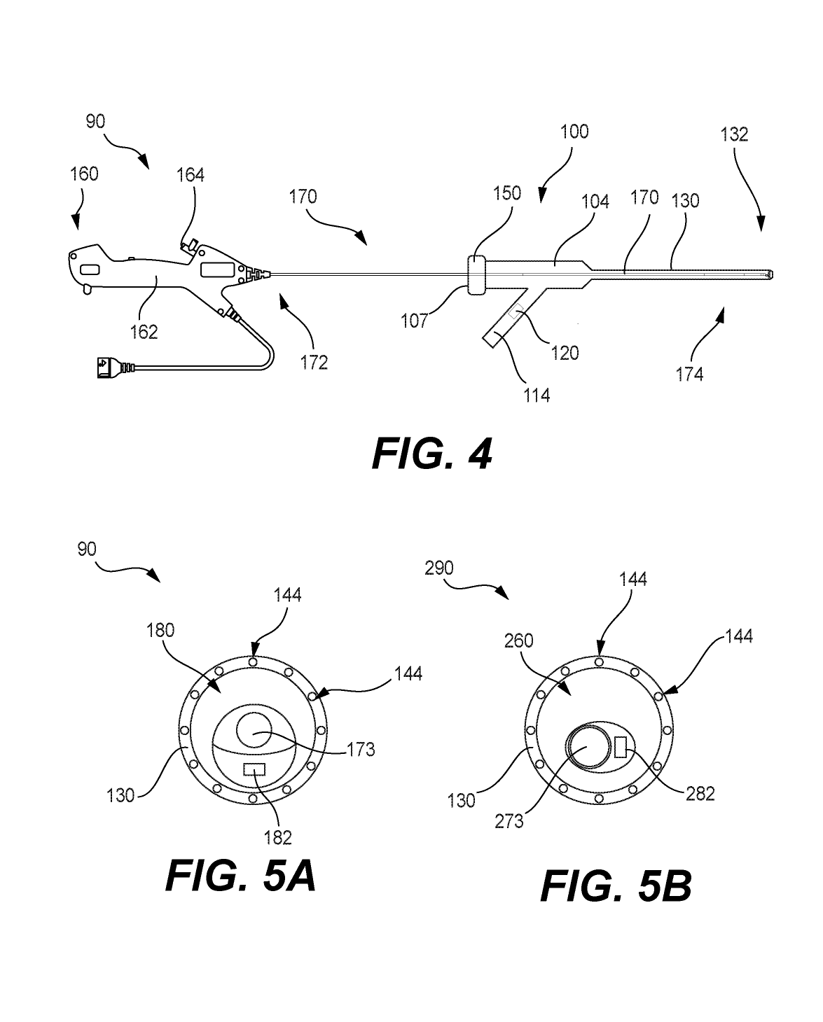

Handle 102 may include a hub 104 with a proximal opening 107 configured to slidably receive a medical instrument. As illustrated in FIG. 4, proximal opening 107 may receive a medical instrument 160 (e.g., a scope) that can be slidably received into lumen 131 of sheath 130. The proximal end of handle 102 (e.g., the proximal end of hub 104) may include a seal 150 around proximal opening 107 to help maintain the position of medical instrument 160 relative to medical device 100 while creating a substantially airtight and/or watertight seal around the inserted instrument. Handle 102 also includes a port 114, which may be a suction port that is in fluid communication with lumen 131 of hub 104. For example, port 114 may be configured to connect to a source of vacuum. In some aspects, a user may connect port 114 to a source of fluid instead in order to deliver fluid through lumen 131 rather than suction.

Port 114 may extend along an axis transverse to the longitudinal axis of hub 104, e.g., port 114 being a side port. Port 114 may include an aperture 120 along its wall to allow a user to control pressure within medical device 100 when connected to a vacuum source. For example, the user may cover and uncover aperture 120 to adjust fluid communication with the external environment and control suction pressure.

FIG. 2 depicts an enlarged view of distal end 132 of sheath 130. Distal end 132 of sheath 130 includes a plurality of light sources 144, e.g., configured to emit light radially outward of sheath 130 to illuminate target tissue or the interior of a body lumen during a medical procedure. Each light source 144 may be embedded within or coupled to the outer surface of sheath 130 and may be arranged around the perimeter of sheath 130 proximate a distalmost end of distal end 132. The plurality of light sources 144 may be light emitting diodes (LEDs), optical fibers, or other suitable light sources. The characteristics of light sources 144, including, e.g., intensity, wavelength, and modulation, may be selected according to particular procedures or tissue types during a medical procedure. In some aspects, handle 102 may connect to capital equipment that supplies energy to optical fibers and/or electrical connection within a wall of sheath 130 operably coupled to light sources 144.

As illustrated in FIGS. 1-3, sheath 130 may also include a sensor, e.g., pressure sensor 140. Pressure sensor may be embedded within or coupled to the outer surface of sheath 130. For example, pressure sensor 140 may be housed in a small recess, notch, or other suitable opening. Pressure sensor 140 may be positioned adjacent to light sources 144 and/or proximal to light sources 144 at the distal end 132. Pressure sensor 140 may be configured to measure pressure outside medical device 100, e.g., an active area of pressure sensor 140 being in fluid communication with the external environment, outside medical device 100. In some aspects, pressure sensor may be configure to trigger an alarm if the pressure exceeds a predetermined threshold, e.g., informing the user when pressure may post a risk to the subject. Pressure sensor 140 may transfer data wirelessly or via a wired connection within the wall of sheath 130, e.g., to a processor within handle 102 or operably coupled to handle 102. In some aspects, pressure data may be transmitted in real-time.

For example, during a medical procedure, pressure sensor 140 may periodically or continuously measure pressure at a target site. The medical procedure may include removing kidney stones, for example, wherein the pressure sensor 140 measures renal pelvis pressure. The pressure sensor 140 may be useful to maintain a safe pressure range of approximately 20–30 cmH₂O. The pressure sensor 140 may communicate with a processor, whether in external equipment or handle 102, to analyze the pressure measurements and generate instructions or signals based on the data. For example, if pressure exceeds 40 cmH₂O, pressure sensor 140 may trigger an alarm, prompting the user to adjust suction pressure and/or fluid flow. In cases of sudden spikes above 60 cmH₂O, a system 90 that includes medical device (see FIG. 4) may automatically increase suction pressure, e.g., to avoid renal damage. The processor may be configured to relay instructions to halt the procedure, or to initiate aspiration through a medical instrument of system 90 (e.g., medical instrument 160). For example, the working channel 173 of instrument 160 may be used for aspiration to lower pressure at the target site.

As mentioned above, medical instrument 160 may be a scope capable of being introduced into medical device, e.g., to be slidably disposed into lumen 131 of sheath 130. In some aspects, medical device 100 obviates the need for illumination from other sources, such as instrument 160. Accordingly, instrument 160 optionally does not include light source(s). Additionally, or alternatively, instrument 160 does not include sensors, such as a pressure sensor, e.g., given the capabilities of medical device 100. The plurality of light sources 144 and pressure sensor 140 on sheath 130 of medical device provides the option to measure pressure and illuminate a target site during use of medical device 100, while leaving space in instrument 160 for other uses, e.g., a relatively larger working channel.

FIG. 3 shows a cross-sectional view of the distal end 132 of sheath 130. Lumen 131 may be in fluid communication with proximal opening 107 and have an open distal end, allowing insertion of medical instrument 160 from the proximal end of medical device 100 and through sheath 130. Lumen 131 may have a diameter sufficient to accommodate a shaft 170 of medical instrument 160 (FIG. 4). For example, lumen 131 may have a diameter ranging from about 10 mm to about 15 mm, such as about 12 mm to about 15 mm. When shaft 170 extends through lumen 131, space radially outward of shaft 170 may provide for a channel to provide vacuum and/or fluid, e.g., via port 114. Sheath 130 and shaft 170 may be sufficiently flexible to navigate anatomy during procedures.

Each light source 144 may be operably connected to a power source via connections extending through the wall of sheath 130. When light sources 144 include optical fibers, the optical fibers may extend through sheath to handle 102. For example, the wall of sheath 130 may define lumens wherein each optical fiber occupies its own lumen serving as a conduit for the fiber. When light sources include LEDs, wires likewise may extend through lumens of the wall of sheath for connection to a power source coupled to handle 102. Further, pressure sensor 140 may be operably connected to electrical components of handle, e.g., a processor, power source, etc., through the wall of sheath 130.

In some aspects, distal end 132 may include a tapered portion 134 as illustrated in the example shown in FIG. 3. Tapered portion 134 may provide an incline that assists in directing light emitted by light sources 144 outward away from sheath 130. Light sources 144 may be coupled to or embedded within tapered portion 134 and oriented to emit light in the direction radially outward from sheath 130 as indicated by arrows "D" in FIG. 3.

The plurality of light sources 144 may be arranged around the perimeter of distal end 132 of sheath 130. This arrangement may be at regular intervals, e.g., to provide extensive illumination at a target site, while maintaining an unobstructed lumen 131. The number of light sources 144 may range from at least 2 to 10 or more. In some examples, sheath 130 may include at least 4 light sources 144, such as 4 to 20 light sources 144, 5 to 15 light sources 144, or 8 to 10 light sources 144. In at least one example, sheath includes 5 light sources 144 on each circumferential side of distal end 132 (i.e., 10 total as illustrated), spaced at regular intervals with one light source directly opposite another light source.

As mentioned above, medical device 100 may be a component of a system that also includes a medical instrument. FIG. 4 illustrates an exemplary medical system 90 according to aspects of the disclosure. Medical system 90 comprises medical device 100 and medical instrument 160. Medical instrument 160 may be a ureteroscope, endoscope, hysteroscope, bronchoscope, cystoscope, or similar device with a working channel. Medical instrument 160 may include shaft 170 and handle 162. Shaft 170 may be a flexible member adapted to be advanced into a patient.

Shaft 170 extends from a proximal end 172 to a distal end 174, defining at least one working channel 173 extending from proximal end 172 to an opening at distal end 174. In some aspects, shaft 170 may include more than one working channel. As shown in FIG. 4, proximal end 172 of shaft 170 may be coupled to handle 162. When instrument 160 is inserted into sheath 130, and at least distal end 174 of shaft 170 may be radially surrounded by sheath 130.

Handle 162 may include a port 164 that may be configured to introduce and/or remove tools, fluids, or other materials to and from shaft 170 for delivery to a target site. Port 164 is fluidically connected to working channel 173 within shaft 170. Port 164 may accommodate passage of various medical instruments such as laser fibers, forceps, or biopsy tools through working channel 173 of shaft 170. Additionally, an umbilicus may connect to handle 162 to provide fluid, suction, or power to medical instrument 160. The distal end of shaft 170, e.g., a distalmost face 180 of shaft 170 may include an imaging element 182 (e.g., a camera) (FIG. 5A).

Handle 162 may include one or more actuators and a plurality of actuating elements, such as cables or wires, which may extend distally from handle 162 through shaft 170. For example, the actuator(s) may include one or more rotatable knobs that rotate relative to handle 162 to push/pull actuating elements to control movement of shaft 170.

During an exemplary medical procedure, a user may insert sheath 130 of medical device into a bodily lumen of a subject, such as through the urethra, bladder, and ureter to reach the kidney. Optionally, the user may employ the plurality of light sources 144 to help guide navigation of sheath 130. Upon reaching a target site, the user may insert medical instrument 160 through proximal opening 107 and seal 150 of medical device 100. The user then advances shaft 170 through hub 104 of medical device 100, advancing shaft 170 through lumen 131 of sheath 130. As the user continues to advance shaft 170 through lumen 131, the user may position the distal face 180 of shaft 170 at or proximate the distal opening of lumen 131.

Working channel 173 of shaft 170 may be used to deliver fluid and/or tools while imaging element 182 may be used to capture images of the target site. For example, a laser may be advanced through working channel 173 to the target site and operated to break apart stones in the kidney into smaller fragments for removal. Fluid may be introduced through working channel 173 or another channel of shaft 170 to irrigate the target site before, during or after using the laser. The user may apply suction through sheath 130 via suction port 114 to remove stone fragments, fluid, and/or other material from the target site through the channel radially between sheath 130 and shaft 170. Pressure sensor 140 may be used to measure pressure and determine whether pressure becomes too high (e.g., due to the presence of fluid) or too low (e.g., due to suction). The plurality of light sources 144 may be used to illuminate the target site, e.g., to assist in capturing images and navigating medical instrument 160 and/or other tools at the target site during a medical procedure.

FIG. 5A illustrates an end view, looking proximally, of shaft 170 within sheath 130 (e.g., showing the distalmost face 180 of shaft 170 and the distalmost face of sheath 130. As mentioned above, the integration of light sources 144 and pressure sensor 140 into sheath 130 may provide illumination and pressure sensing capability without including those components in medical instrument 160. As a result, working channel 173 can be relatively larger and occupy at least part of the space that would otherwise include a sensor and light sources.

FIG. 5B depicts an end view of another exemplary medical system 290 that includes medical device 100 and a different medical instrument 260 than that of medical system 90. Medical instrument 260 may include any of the features of medical instrument 160 unless otherwise specified. As shown, medical instrument 260 has a relatively larger working channel 273, an imaging device 282 at the distalmost face of its shaft. The shaft of medical instrument 260 has a generally oval cross-section rather than the circular cross-section of medical instrument 160. Both FIGS. 5A and 5B illustrate the capability to use medical instruments with larger working channels and without light sources or sensors, while having the capability of illumination and pressure measurements via medical device 100.

The medical devices herein may be used with a relatively more compact medical instrument (e.g., medical instrument 160 or medical instrument 260), which may help to mitigate risks associated with high intrarenal pressure. The diameter ratio between the medical instrument and sheath 130 may influence pressure within a body lumen, for example, and a lower ratio may help pressure regulation and management.

While principles of the disclosure are described herein with reference to illustrative aspects for particular medical uses and procedures, the disclosure is not limited thereto. Those having ordinary skill in the art and access to the teachings provided herein will recognize additional modifications, applications, aspects, and substitution of equivalents all fall in the scope of the aspects described herein. Accordingly, the disclosure is not to be considered as limited by the foregoing description.

1. A medical device comprising:
a sheath defining a lumen extending to a distal end of the sheath, wherein the lumen is configured to slidably receive a medical instrument;
a pressure sensor embedded within or coupled to an outer surface of the sheath; and
a plurality of light sources arranged about a perimeter of the distal end of the sheath, wherein the pressure sensor is proximal to the plurality of light sources.

2. The medical device of claim 1, wherein each light source of the plurality of light sources comprises a light-emitting diode (LED) or an optical fiber.

3. The medical device of claim 1, wherein the distal end of the sheath includes a tapered portion and the plurality of light sources is disposed on the tapered portion.

4. The medical device of claim 1, wherein the plurality of light sources includes at least four light sources.

5. The medical device of claim 1, wherein the plurality of light sources includes at least ten light sources arranged at regular intervals.

6. The medical device of claim 1, wherein the lumen has a diameter ranging from about 10 mm to about 15 mm.

7. The medical device of claim 1, wherein each light source of the plurality of light sources includes an optical fiber extending through a wall of the sheath.

8. The medical device of claim 1, wherein the pressure sensor is configured to trigger an alarm when measuring a pressure outside the medical device that exceeds a predetermined threshold.

9. The medical device of claim 1, further comprising a handle coupled to or integrated with the sheath, wherein the handle includes a suction port in fluid communication with the lumen.

10. The medical device of claim 9, wherein the suction port includes an aperture configured to control pressure in the medical device when the suction port is coupled to a source of vacuum.

11. The medical device of claim 1, wherein the sheath is flexible and configured to navigate through a urinary system of a subject.

12. The medical device of claim 1, wherein the pressure sensor is adjacent to at least one light source of the plurality of light sources.

13. The medical device of claim 1, wherein the lumen extends an entire length of the sheath.

14. A system comprising the medical device of claim 1 and a medical instrument, wherein the medical instrument is slidably disposed in the lumen, and wherein the medical instrument is a scope that does not include a light source.

15. The system of claim 14, wherein the scope is a ureteroscope.

16. A medical device comprising:
a sheath defining a lumen extending to a distal end of the sheath, wherein the lumen is configured to slidably receive a ureteroscope;
a pressure sensor embedded within or coupled to an outer surface of the sheath;
a plurality of light sources including at least four light sources arranged about a perimeter of the distal end of the sheath; wherein the pressure sensor is proximal to the plurality of light sources; and
a handle coupled to or integrated with the sheath, wherein the handle includes a suction port in fluid communication with the lumen.

17. The medical device of claim 16, wherein each light source of the plurality of light sources comprises a light-emitting diode (LED) or an optical fiber.

18. The medical device of claim 16, wherein the distal end of the sheath includes a tapered portion and the plurality of light sources is disposed on the tapered portion.

19. The medical device of claim 16, wherein the lumen has a diameter ranging from about 10 mm to about 15 mm.

20. A system comprising:
a medical device comprising:
a sheath defining a lumen extending an entire length of the sheath, wherein the lumen is configured to slidably receive a medical instrument;
a pressure sensor embedded within or coupled to an outer surface of the sheath; and
a plurality of light sources arranged about a perimeter of a distal end of the sheath; wherein the pressure sensor is proximal to the plurality of light sources; and
the medical instrument, wherein the medical instrument is a scope that does not include a light source.
